(19) United States
(12) Patent Application Publication
(10) Pub. No.: US 2026/0233208 A1
(43) Pub. Date: Aug. 13, 2026

(54) METALLOSILICATE CATALYST SOLVENTS

(71) Applicant: Dow Global Technologies LLC, Midland, MI (US)

(72) Inventors: Kimberly Dinh, Midland, MI (US); Wen-Sheng Lee, Midland, MI (US)

(21) Appl. No.: 19/153,650

(22) PCT Filed: Feb. 6, 2024
(86) PCT No.: PCT/US2024/014549

§ 371 Date: Aug. 5, 2025

Related U.S. Application Data
(60) Provisional application No. 63/446,914, filed on Feb. 20, 2023.

(51) Int. Cl. B01J 29/70; C07C 41/06; C07C 43/13

(52) U.S. Cl. CPC B01J 29/7007; C07C 41/06; C07C 43/13

(57) ABSTRACT

A method of generating an alkylene glycol monoalkyl ether includes the steps of contacting an olefin, an alcohol, a metallosilicate catalyst, and a solvent blend, wherein the solvent blend comprises from 30 wt % to 90 wt % of an oxygenated solvent based on a total weight of the solvent blend and 10 wt % to 70 wt % of a non-oxygenated solvent based on a total weight of the solvent blend; and generating the alkylene glycol monoalkyl ether.

### BACKGROUND

### Field of the Invention

The present disclosure generally relates to metallosilicate catalysts and more specifically to solvent blends used in conjunction with metallosilicate catalysts.

### INTRODUCTION

Production of secondary alcohol ethoxylate surfactants can be carried out by the catalyzed ethoxylation of (poly)alkylene glycol monoalkyl ether (“monoalkyl ether”). The monoalkyl ether is formed from an olefin and a (poly)alkylene glycol via an etherification reaction using metallosilicate catalysts. The etherification reaction also produces (poly)alkylene glycol dialkyl ether (“dialkyl ether”). The selectivity of a catalyst or a reaction indicates the relative proportions of reaction products produced. Typically, a high selectivity of the desired reaction product is advantageous in maximizing the output of the reaction. Metallosilicate catalysts offer a selectivity for monoalkyl ether of greater than 80% which is advantageous as dialkyl ether is deleterious to properties of the secondary alcohol ethoxylate surfactants.

Another important metric for the etherification reaction is olefin conversion. Olefin conversion measures the amount of olefin that is converted in the reaction. Similarly to selectivity, it is advantageous to maximize the olefin conversion to maximize the output of the reaction. It is difficult to achieve concurrent high monoalkyl ether selectivity and high olefin conversion as monoalkyl ether is an intermediate for the undesired dialkyl ether formation. In other words, when olefin conversion is pushed higher, a higher dialkyl ether content is generated and less monoalkyl ether will remain (i.e., lower selectivity). Without using a solvent, olefin conversion of 20% to 40% and monoalkyl ether selectivity of 70% to 75% can be obtained. The relatively low conversion and low selectivity becomes a hurdle to implementing the etherification reaction at a large scale due to excessive dialkyl ether production. Ideally, the etherification reaction used in the production of monoalkyl ether should have greater than 40% olefin conversion and a monoalkyl ether selectivity of greater than 75% in order to be considered successful.

Attempts at utilizing solvents to improve the selectivity and olefin conversion have been attempted. For example, U.S. Pat. Nos. 5,741,948 and 6,417,408 both disclose that reaction between an olefin and (poly)alkylene glycol can be conducted in the presence or absence of a solvent such as “nitromethane, nitroethane, nitrobenzene, dioxane, ethylene glycol dimethyl ether, sulfolane, benzene, toluene, xylene, hexane, cyclohexane, decane, paraffin, etc.” U.S. Pat. Nos. 5,741,948 and 6,417,408 do not indicate a preference for type of solvent or disclose the mixing of different solvents. Similarly, US20220274903A1 discloses the use of oxygenated solvents in the production of monoalkyl ether, but makes clear through comparative examples 5 and 8 that hexane as a solvent does not offer monoalkyl ether selectivity or olefin conversion advantages. WO2023278187 also discloses the use of cresols and other oxygenated solvents in connection with the production of monoalkyl ether.

Despite the apparent benefits of using some types of solvents, the addition of more materials to the reaction adds greater cost. For example, oxygenated solvents such as those described in the above-noted prior art are relatively expensive and can make the commercial production of monoalkyl ether prohibitively expensive.

In view of the foregoing, it would be surprising to discover a method of generating an alkylene glycol monoalkyl ether that has greater than 40% olefin conversion and a monoalkyl ether selectivity of greater than 75% and also is financially viable.

### SUMMARY OF THE DISCLOSURE

The inventors of the present application have discovered a method of generating an alkylene glycol monoalkyl ether that has greater than 40% olefin conversion and a monoalkyl ether selectivity of greater than 75% and also is financially viable.

The invention is the result of discovering that utilizing a solvent blend comprising both oxygenated and non-oxygenated solvents in the etherification reaction achieves a greater than 40% olefin conversion and a monoalkyl ether selectivity of greater than 75% while remaining financially viable. While oxygenated solvents are typically expensive and non-oxygenated solvents cannot on their own achieve both the desired olefin conversion or selectivity, it has been discovered that a solvent blend comprising from 30 wt % to 90 wt % of an oxygenated solvent based on a total weight of the solvent blend and 10 wt % to 70 wt % of a non-oxygenated solvent based on a total weight of the solvent blend can achieve all the desired objectives. Further, such combinations surprisingly demonstrate minimal to no decrease in the olefin conversion and selectivity despite the addition of the non-oxygenated solvent.

According to a first feature of the present disclosure, a method of generating an alkylene glycol monoalkyl ether comprises the steps of contacting an olefin, an alcohol, a metallosilicate catalyst, and a solvent blend, wherein the solvent blend comprises from 30 wt % to 90 wt % of an oxygenated solvent based on a total weight of the solvent blend and 10 wt % to 70 wt % of a non-oxygenated solvent based on a total weight of the solvent blend; and generating the alkylene glycol monoalkyl ether.

According to a second feature of the present disclosure, the metallosilicate catalyst is an aluminosilicate compound having a crystal lattice that has had one or more metal elements substituted in the crystal lattice for a silicon atom.

According to a third feature of the present disclosure, the olefin comprises a C₁₂-C₁₄ alpha-olefin.

According to a fourth feature of the present disclosure, the alcohol is monoethylene glycol, diethylene glycol, glycerol or combinations thereof.

According to a fifth feature of the present disclosure, an alcohol to olefin molar ratio is from 0.5 to 15.

According to a sixth feature of the present disclosure, the solvent blend is from 10 wt % to 80 wt % of the total weight of the combined olefin, alcohol, and solvent blend.

According to a seventh feature of the present disclosure, the solvent blend comprises from 40 wt % to 60 wt % of the non-oxygenated solvent based on a total weight of the solvent blend.

According to an eight feature of the present disclosure, the oxygenated solvent is selected from the group consisting of cresols, o-cresol, m-cresol, p-cresol, phenol, guaiacol, methyl benzoate, dimethyl phthalate, diglyme, triglyme, diethyl phthalate, dibutyl phthalate, ethyl benzoate, trimethyl 1,2,4-benzenetricarboxylate, glyme, dimethoxybenzene and combinations thereof.

According to a ninth feature of the present disclosure, the non-oxygenated solvent is selected from the group consisting of xylenes, m-xylene, p-xylene, o-xylene, benzene, toluene, hexane, heptane, octane, nonane, undecane, dodecane, tridecane, tetradecane, pentadecane, hexadecane, cyclohexane, cycloheptane, cyclooctane, linear C₆ to C₁₆ alkanes, cyclic C₆ to C₁₆ alkanes and combinations thereof.

According to a tenth feature of the present disclosure, the non-oxygenated solvent is xylene and the oxygenated solvent is cresols.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

Test methods refer to the most recent test method as of the priority date of this document unless a date is indicated with the test method number as a hyphenated two-digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut für Normung; and ISO refers to International Organization for Standards.

IUPAC codes describing Crystal structures as delineated by the Structure Commission of the International Zeolite Association refer to the most recent designation as of the priority date of this document unless otherwise indicated.

As used herein, the term weight percent (“wt %”) designates the percentage by weight a component is of a total weight of an indicated composition.

As used herein, a “CAS number” is the chemical services registry number assigned by the Chemical Abstracts Service.

### Method

The method of the present invention is directed to the use of a solvent blend in a metallosilicate catalyzed reaction of alcohol and olefin. The method may comprise steps of (a) contacting an olefin, an alcohol, a metallosilicate catalyst and a solvent blend and (b) generating an alkylene glycol monoalkyl ether.

### Olefin

The olefin used in the method may be linear, branched, acyclic, cyclic, or mixtures thereof. The olefin may have from 5 carbons to 30 carbons (i.e., C₅-C₃₀). The olefin may have 5 carbons or greater, or 6 carbons or greater, or 7 carbons or greater, or 8 carbons or greater, or 9 carbons or greater, or 10 carbons or greater, or 11 carbons or greater, or 12 carbons or greater, or 13 carbons or greater, or 14 carbons or greater, or 15 carbons or greater, or 16 carbons or greater, or 17 carbons or greater, or 18 carbons or greater, or 19 carbons or greater, or 20 carbons or greater, or 21 carbons or greater, or 22 carbons or greater, or 23 carbons or greater, or 24 carbons or greater, or 25 carbons or greater, or 26 carbons or greater, or 27 carbons or greater, or 28 carbons or greater, or 29 carbons or greater, while at the same time, 30 carbons or less, or 29 carbons or less, or 28 carbons or less, or 27 carbons or less, or 26 carbons or less, or 25 carbons or less, or 24 carbons or less, or 23 carbons or less, or 22 carbons or less, or 21 carbons or less, or 20 carbons or less, or 19 carbons or less, or 18 carbons or less, or 17 carbons or less, or 16 carbons or less, or 15 carbons or less, or 14 carbons or less, or 13 carbons or less, or 12 carbons or less, or 11 carbons or less, or 10 carbons or less, or 9 carbons or less, or 8 carbons or less, or 7 carbons or less, or 6 carbons or less.

The olefin may include alkenes such as alpha (α) olefins, internal disubstituted olefins, or cyclic structures (e.g., C₃-C₁₂ cycloalkene). α olefins include an unsaturated bond in the α-position of the olefin. Suitable α olefins may be selected from the group consisting of propylene, 1-butene, 1-hexene, 4-methyl-1-pentene, 1-heptene, 1-octene, 1-decene, 1-dodecene, 1-tetradecene, 1-hexadecene, 1-octadecene, 1-icosene, 1-docosene and combinations thereof. Internal disubstituted olefins include an unsaturated bond not in a terminal location on the olefin. Internal olefins may be selected from the group consisting of 2-butene, 2-pentene, 2-hexene, 3-hexene, 2-heptene, 3-heptene, 2-octene, 3-octene, 4-octene, 2-nonene, 3-nonene, 4-nonene, 2-decene, 3-decene, 4-decene, 5-decene and combinations thereof. Other exemplary olefins may include butadiene and styrene.

Examples of suitable commercially available olefins include NEODENE™ 6-XHP, NEODENE™ 8, NEODENE™ 10, NEODENE™ 12, NEODENE™ 14, NEODENE™ 16, NEODENE™ 1214, NEODENE™ 1416, NEODENE™ 16148 from Shell, The Hague, Netherlands.

### Alcohol

The alcohol utilized in the method may comprise a single hydroxyl group, may comprise two hydroxyl groups (i.e., a glycol) or may comprise three hydroxyl groups. The alcohol may include 1 carbon or greater, or 2 carbons or greater, or 3 carbons or greater, or 4 carbons or greater, or 5 carbons or greater, or 6 carbons or greater, or 7 carbons or greater, or 8 carbons or greater, or 9 carbons or greater, while at the same time, 10 carbons or less, or 9 carbons or less, or 8 carbons or less, or 7 carbons or less, or 6 carbons or less, or 5 carbons or less, or 4 carbons or less, or 3 carbons or less, or 2 carbons or less. The alcohol may be selected from the group consisting of methanol, ethanol, monoethylene glycol, diethylene glycol, propylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, 1,4-cyclohexanemethanediol, glycerol and/or combinations thereof. According to various examples, the alcohol is a (poly)alkylene glycol such as monoethylene glycol, diethylene glycol, propylene glycol and triethylene glycol.

A molar ratio of alcohol to olefin in the method may be from be 20:0.5 or less, or 20:1 or less, or 15:1 or less, or 10:1 or less, or 9:1 or less, or 8:1 or less, or 7:1 or less, or 6:1 or less, or 5:1 or less, or 4:1 or less, or 3:1 or less, or 2:1 or less, or 0.2:1 or less, while at the same time, 0.1:1 or greater, or 1:1 or greater, or 1:2 or greater, or 1:3 or greater, or 1:4 or greater, or 1:5 or greater, or 1:6 or greater, or 1:7 or greater, or 1:8 or greater, or 1:9 or greater, or 1:10 or greater, or 1:15 or greater, or 1:20 or greater of 0.5:20 or greater. In specific examples, the molar ratio of the alcohol to the olefin may be from 2.0 to 15, or from 2.0 to 8. Molar ratio is calculated by dividing the number of moles of alcohol present by the number of moles of olefin present.

### Solvent Blend

The solvent blend is contacted with the olefin, the alcohol and the metallosilicate catalyst in order to facilitate a chemical reaction. The solvent blend comprises an oxygenated solvent and a non-oxygenated solvent. As used herein, the term “oxygenated solvent” means a compound comprising carbon, hydrogen and oxygen atoms that solubilize one or more of the alcohol and the olefin. As used herein, the term “non-oxygenated solvent” means a compound which does not include an oxygen atom and which solubilize one or more of the alcohol and the olefin.

The solvent blend may be from 0.1 wt % to 85 wt % of the combined olefin, alcohol and solvent blend weight. The solvent blend may be 0.1 wt % or greater, or 0.5 wt % or greater, or 1 wt % or greater, or 5 wt % or greater, or 10 wt % or greater, or 15 wt % or greater, or 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, or 75 wt % or greater, or 80 wt % or greater, while at the same time, 85 wt % or less, or 80 wt % or less, or 75 wt % or less, or 70 wt % or less, 65 wt % or less, or 60 wt % or less, 55 wt % or less, or 50 wt % or less, 45 wt % or less, or 40 wt % or less, 35 wt % or less, or 30 wt % or less, 25 wt % or less, or 20 wt % or less, 15 wt % or less, or 10 wt % or less, 5 wt % or less, or 1 wt % or less, or 0.5 wt % or less of the combined olefin, alcohol and solvent blend weight.

The solvent blend comprises from 30 wt % to 90 wt % of the oxygenated solvent based on a total weight of the solvent blend and 10 wt % to 70 wt % of a non-oxygenated solvent based on a total weight of the solvent blend. For example, the solvent blend may comprise 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, or 75 wt % or greater, or 80 wt % or greater, or 85 wt % or greater, while at the same time, 90 wt % or less, or 85 wt % or less, or 80 wt % or less, or 75 wt % or less, or 70 wt % or less, or 65 wt % or less, or 60 wt % or less, or 55 wt % or less, or 50 wt % or less, or 45 wt % or less, or 40 wt % or less, or 35 wt % or less of the oxygenated solvent based on the total weight of the solvent blend. The solvent blend may comprise 10 wt % or greater, or 15 wt % or greater, or 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, while at the same time, 70 wt % or less, or 65 wt % or less, or 60 wt % or less, or 55 wt % or less, or 50 wt % or less, or 45 wt % or less, or 40 wt % or less, or 35 wt % or less, or 30 wt % or less, or 25 wt % or less, or 20 wt % or less, or 15 wt % or less of the non-oxygenated solvent based on the total weight of the solvent blend.

The oxygenated solvent is selected from the group consisting of cresols, o-cresol, m-cresol, p-cresol, phenol, guaiacol, methyl benzoate, dimethyl phthalate, diglyme, triglyme, diethyl phthalate, dibutyl phthalate, ethyl benzoate, trimethyl 1,2,4-benzenetricarboxylate, glyme, dimethoxybenzene and combinations thereof. As used herein, the term “cresols” means a blend of the o-cresol, p-cresol and m-cresol isomers.

The non-oxygenated solvent is selected from the group consisting of xylenes, m-xylene, p-xylene, o-xylene, benzene, toluene, hexane, heptane, octane, nonane, undecane, dodecane, tridecane, tetradecane, pentadecane, hexadecane, cyclohexane, cycloheptane, cyclooctane, linear C₆ to C₁₆ alkanes, cyclic C₆ to C₁₆ alkanes and combinations thereof. As used herein, the term “xylenes” refers to a blend of xylene isomers m-xylene, p-xylene and o-xylene. In a specific example, the oxygenated solvent is cresols and the non-oxygenated solvent is xylenes.

### Metallosilicate Catalyst

As used herein the term “metallosilicate catalyst” is an aluminosilicate (commonly referred to as a zeolite) compound having a crystal lattice that has had one or more metal elements substituted in the crystal lattice for a silicon atom. The crystal lattice of the metallosilicate catalyst form cavities and channels inside where cations, water and/or small molecules may reside. The substitute metal element may include one or more metals selected from the group consisting of B, Al, Ga, In, Ge, Sn, P, As, Sb, Sc, Y, La, Ti, Zr, V, Cr, Mn, Pb, Pd, Pt, Au, Fe, Co, Ni, Cu, Zn. The metallosilicate catalyst may be substantially free of Hf. According to various examples, the metallosilicate may have a silica to alumina molar ratio of from 5:1 to 1,500:1 as measured using Neutron Activation Analysis. The silica to alumina molar ratio may be from 5:1 to 1,500:1, or from 10:1 to 500:1, or from 10:1 to 400:1, or from 10:1 to 300:1 or from 10:1 to 200:1. Such a silica to alumina molar ratio may be advantageous in providing a metallosilicate catalyst with an appropriate hydrophobic selectivity that adsorb non-polar organic molecules.

The metallosilicate catalyst may have one or more ion-exchangeable cations outside the crystal lattice. The ion-exchangeable cation may include H⁺, Li⁺, Na⁺, Rb⁺, Cs⁺, Mg²⁺, Ca²⁺, Sr²⁺, Ba²⁺, Sc³⁺, Y³⁺, La³⁺, R₄N⁺, R₄P⁺ (where R is H or alkyl).

The metallosilicate catalyst may take a variety of crystal structures. Specific examples of the metallosilicate catalyst structures include MFI (e.g. ZSM-5), MEL (e.g. ZSM-11), BEA (e.g. β-type zeolite), FAU (e.g. Y-type zeolite), MOR (e.g. Mordenite), MTW (e.g. ZSM-12), and LTL (e.g. Linde L), as described using IUPAC codes in accordance with nomenclature by the Structure Commission of the International Zeolite Association.

The crystalline frameworks of metallosilicate catalyst are represented by networks of molecular-sized channels and cages comprised of corner-shared tetrahedral [TO₄] (T=Si or Al) primary building blocks. A negative charge can be introduced onto the framework via the isomorphous substitution of a framework tetravalent silicon by a trivalent metal (e.g., aluminum) atom. The overall charge neutrality is then achieved by the introduction of cationic species compensating for the resulting negative lattice charge. When such a charge-compensation is provided by protons, Brønsted acid sites are formed rendering the resulting H-forms of zeolites strong solid Brønsted acids.

The metallosilicate catalysts may be used in the method in a variety of forms. For example, the metallosilicate catalysts may be powdered (e.g., particles having a longest linear dimension of less than 100 micrometers), granular (e.g., particles having a longest linear dimension of 100 micrometers or greater), or molded articles (e.g., pellets or extrudates) of powdered and/or granular metallosilicate catalysts.

The metallosilicate catalysts may have a surface area of 100 m²/g or greater, or 200 m²/g or greater, or 300 m²/g or greater, or 400 m²/g or greater, or 500 m²/g or greater, or 600 m²/g or greater, or 700 m²/g or greater, or 800 m²/g or greater, or 900 m²/g or greater, while at the same time, 1000 m²/g or less, or 900 m²/g or less, or 800 m²/g or less, or 700 m²/g or less, or 600 m²/g or less, or 500 m²/g or less, or 400 m²/g or less, or 300 m²/g or less, or 200 m²/g or less. Surface area is measured according to ASTM D4365-19.

Metallosilicate catalysts can be synthesized by hydrothermal synthesis methods. For example, the metallosilicate catalysts can be synthesized from heating a composition comprising a silica source (e.g., silica sol, silica gel, and alkoxysilanes), a metal source (e.g., metal sulfates, metal oxides, metal halides, etc.), and a quaternary ammonium salt such as a tetraethylammonium salt or tetrapropylammonium to a temperature of about 100° C. to about 175° C. until a crystal solid forms. The resulting crystal solid is then filtered off, washed with water, and dried, and then calcined at a temperature form 350° C. to 600° C.

Examples of suitable commercially available, metallosilicate catalysts include CP814E, CP814C, CP811EL, CP811C-300, CBV 712, CBV 720, CBV 760, CBV 2314, CBV 10A from ZEOLYST INTERNATIONAL™ of Conshohocken, PA.

### Generating Monoalkyl Ether

Contacting the olefin, alcohol, metallosilicate catalyst and solvent blend results in the generation of an alkylene glycol monoalkyl ether. The chemical reaction between the olefin and the alcohol is catalyzed by the metallosilicate catalyst in a reactor to generate the monoalkyl ether. Various monoalkyl ethers may be produced for different applications by varying which olefin is utilized and/or by varying which alcohol is utilized. Monoalkyl ethers are utilized for a number of applications such as solvents, surfactants, and chemical intermediates, for instance.

The reaction of the olefin and the alcohol may take place at from 50° C. to 300° C. or from 100° C. to 200° C. In a specific example the reaction may be carried out at 150° C. In another specific example, the reaction may be carried out at 135° C. In another specific example, the reaction may be carried out at a temperature of 120° C. to 150° C. Reaction of the olefin and the alcohol may be carried out in a batch reactor, continuous reactor, or fixed-bed reactor. In operation of the chemical reaction, the Brønsted acid sites of the metallosilicate catalyst catalyze the etherification of the olefin to the alcohol through an addition type reaction. The reaction of the olefin and the alcohol produces the monoalkyl ether.

The addition reaction of the olefin to the glycol may form not only monoalkyl ether but also the dialkyl ether. The metallosilicate catalyst may exhibit a selectivity to produce alkylene monoalkyl ether, but not dialkyl ether. The monoalkyl ether selectivity may be 70% or greater, or 75% or greater, or 80% or greater, or 85% or greater, or 90% or greater, or 95% or greater or 99% or greater, while at the same time, 100% or less, or 95% or less, or 90% or less, or 85% or less, or 80% or less, or 75% or less. The dialkyl ether selectivity may be 0% or greater, or 2% or greater, or 4% or greater, or 6% or greater, or 8% or greater, or 10% or greater, or 12% or greater, or 14% or greater, or 16% or greater, or 18% or greater, while at the same time, 20% or less, or 18% or less, or 16% or less, or 14% or less, or 12% or less, or 10% or less, or 8% or less, or 6% or less, or 4% or less, or 2% or less.

A monoalkyl ether yield is calculated by multiplying the amount of olefin conversion by the monoalkyl ether selectivity. The alkylene glycol monoalkyl ether yield may be 10% or greater, or 15% or greater, or 20% or greater, or 25% or greater, or 30% or greater, or 35% or greater, while at the same time, 40% or less, or 35% or less, or 30% or less, or 25% or less, or 20% or less, or 15% or less. Monoalkyl ether yield is a measure of the catalytic activity and selectivity and is a good measure of the production rate of the metallosilicate catalyst.

During the reaction of the olefin and the alcohol, the catalyst becomes fouled and results in the catalyst deactivating (i.e., lost etherification activity >90%) within hours.

### EXAMPLES

### Materials

Below is a listing of the materials used in the formation of the inventive examples (“IE”) and the comparative examples (“CE”).
Catalyst is a metallosilicate catalysts defined by a BEA structure and having a silica to alumina molar ratio of 25:1 and a surface area of 680 m²/g, that is commercially available as CP814E from ZEOLYST INTERNATIONAL™ of Conshohocken, PA.
Olefin is 1-Dodecene that is commercially available as NEODENE™ 12 from the SHELL™ group of The Hague, Netherlands.
Monoethylene Glycol is polyester grade with a CAS Number of 107-21-1 and is sourced from The Dow Chemical Company, Midland, Michigan.
Cresols is a mixture of O-cresol, P-cresol and M-cresol isomers in up to 8 wt % phenol and is available from SIGMA ALDRICH™ St. Louis, Missouri.
M-Cresol is 3-methylphenol having a CAS number of 108-39-4 and is commercially available.
Xylene is a mixture of o-xylene, m-xylene, and p-xylene with ethylbenzene as a cosolvent and is available from SIGMA ALDRICH™ St. Louis, Missouri.
O-xylene is 1,2-Dimethylbenzene and is commercially available from SIGMA ALDRICH™ St. Louis, Missouri.
Decane is a non-oxygenated solvent and is commercially available from SIGMA ALDRICH™ St. Louis, Missouri.
Methyl benzoate is a non-oxygenated solvent having a CAS number of 93-58-3 and is commercially available from SIGMA ALDRICH™ St. Louis, Missouri.
Diglyme is bis(2-methoxyethyl) ether having a CAS number of 111-96-6 and is commercially available from SIGMA ALDRICH™ St. Louis, Missouri.
Guaiacol is 2-Methoxyphenol having a CAS number of 90-05-1 and is commercially available from SIGMA ALDRICH™ St. Louis, Missouri.
Toluene is a non-oxygenated solvent and is commercially available from SIGMA ALDRICH™ St. Louis, Missouri.
Benzene is a non-oxygenated solvent and is commercially available from SIGMA ALDRICH™ St. Louis, Missouri.

### Test Methods

### Gas Chromatography Samples

Prepare gas chromatography samples by mixing 100 μL of the example with 10 mL of gas chromatography solution that was prepared by addition of 1 mL of hexadecane in 1 L of ethyl acetate. Analyze the sample using an Agilent 7890B gas chromatography instrument. Determine the total amount of 1-dodecene derived species, which includes monoalkyl ether, dialkyl ether and 2-dodecanol, total amount of dodecene, which includes 1-dodecene and all non 1-dodecene other C₁₂ isomers. Table 1 provides the relevant gas chromatography instrument parameters.

TABLE 1

| Chromatograph: | Agilent 7890 Series GC | |
|---|---|---|
| Column: | Agilent HP88, 100 m × 0.25 mm × 0.20 um | |
| Detector | FID | |
| Oven: | 50° C. - 7 min - 6° C./min - 260° C. - 1 min | |
| Injector: | 250° | C. |
| Detector: | 300° | C. |
| Carrier: | Helium 2.0 mL/min constant flow mode | |
| Split ratio: | 10 | |
| Make-Up: | Nitrogen 25 mL/min | |
| Air: | 400 | mL/min |
| Hydrogen: | 40 | mL/min |

| | |
|---|---|
| Inlet Liner: | Restek PN 23305.5 Sky Precision Liner |
| | with wool |
| Sample Size: | 1 μL |
| GC vial rinsing solvent: | ethyl acetate |

### Olefin Conversion

Calculate the percent olefin conversion by dividing the total amount of dodecene derived species by the summation of total amount of dodecene derived species and the amount of dodecene. Multiply the quotient by 100.

### Monoalkyl Ether Selectivity

Calculate the percent monoalkyl ether selectivity by dividing the total amount of monoalkyl ether by the total amount of dodecene derived species. Multiply the quotient by 100.

### Monoether Yield

Calculate the monoalkyl ether yield by multiplying the olefin conversion value by the monoalkyl ether selectivity value.

### Sample Preparation and Results

The etherification reaction was carried out in either a vial reactor or a Parr reactor. The reactants used were 1-dodecene, ethylene glycol, the indicated solvent(s) and the catalyst. IE6 and IE7 were the only examples run in the Parr reactor. The vial reactor was a 40 mL vial that had the reactants added along with a tumbling disc stirrer. For vial reactors, the reactor included 0.75 g Beta zeolite and 15 grams of reaction mixture (i.e., ethylene glycol, 1-dodecene, and solvents). The vial reactors were then placed in a device that both heated the vial reactor to the desired temperature as well as produced stirring in a tumbling style by the stir bar. The Parr reactor was performed using a 10× scale as that was used in a vial reactor. The Parr reactor included 7.5 g Beta zeolite, 150 g reaction mixture (i.e., ethylene glycol, 1-dodecene, and solvents). The Parr reactors were agitated using an overhead stirrer. For the Parr reactor runs, the reaction was heated up to 135° C. in an hour and held at 135° C. for 3 hours. For the vial reactors runs, the vial reactors were placed in the heating instrument that was preheated at 135° C. and the reaction was held for 3 hours.

Table 1 provides the composition and performance of the inventive and comparative examples. In Table 1, “N—O” is the non-oxygenated solvent and “OXY Solvent” is the oxygenated solvent.

TABLE 1

| | | Ethylene | | | | N—O | | | |
|---|---|---|---|---|---|---|---|---|---|
| | | Glycol/1- | Total Solvent | | | Solvent:OXY | Olefin | Monoether | |
| | Time | Dodecene | Concentration | N—O | OXY | Solvent | Conversion | Selectivity | Monoether |
| EX. | (h) | Ratio | (wt %) | Solvent | Solvent | Ratio | (%) | (%) | Yield (%) |
| CE1 | 3 | 3 | — | — | — | — | 35.4 | 87 | 30.8 |
| CE2 | 3 | 3 | 50 | Xylenes | — | — | 34.8 | 84.8 | 29.5 |
| CE3 | 3 | 3 | 50 | Decane | — | — | 27.8 | 88.5 | 24.6 |
| CE4 | 3 | 3 | 50 | — | M-Cresol | — | 68.2 | 90 | 61.3 |
| CE5 | 3 | 3 | 71 | — | Diglyme | — | 40.3 | 91.0 | 36.7 |
| CE6 | 3 | 3 | 50 | — | Methyl | — | 56.9 | 84.8 | 48.3 |
| | | | | | Benzoate | | | | |
| CE7 | 3 | 3 | 50 | — | Guaiacol | 50:50 | 73.0 | 86.2 | 62.9 |
| IE1 | 3 | 3 | 71 | Xylenes | Diglyme | 50:50 | 40.4 | 87.9 | 35.6 |
| IE2 | 3 | 3 | 50 | Xylenes | Methyl | 50:50 | 47.0 | 81.9 | 38.5 |
| | | | | | Benzoate | | | | |
| IE3 | 3 | 3 | 50 | Xylenes | Guaiacol | 50:50 | 48.5 | 81 | 39 |
| IE4 | 3 | 3 | 50 | Xylenes | Cresols | 50:50 | 60.4 | 84.9 | 51.3 |
| IE5 | 3 | 3 | 50 | Decane | Cresols | 50:50 | 42.4 | 78.9 | 33.4 |
| IE6 | 3 | 3 | 50 | Toluene | M-Cresol | 50:50 | 63.9 | 85.0 | 54.0 |
| IE7 | 3 | 3 | 50 | Benzene | M-Cresol | 50:50 | 68.4 | 86.0 | 59.0 |
| IE8 | 3 | 3 | 30 | O-Xylene | Cresols | 50:50 | 53.0 | 77.4 | 41.0 |
| IE9 | 3 | 3 | 50 | Xylenes | Cresols | 25:75 | 65.1 | 85.9 | 56.0 |
| CE8 | 3 | 3 | 50 | Xylenes | Cresols | 75:25 | 39.5 | 84.0 | 33.2 |
| IE10 | 3 | 6 | 50 | Xylenes | Cresols | 50:50 | 65.2 | 91.9 | 60.0 |
| IE11 | 5 | 3 | 10 | Xylenes | Cresols | 50:50 | 51.1 | 77.5 | 38.4 |

Referring now to Table 1, it is clear from IE1-IE11 that the use of a solvent blend comprises from 30 wt % to 90 wt % of an oxygenated solvent based on a total weight of the solvent blend and 10 wt % to 70 wt % of a non-oxygenated solvent based on a total weight of the solvent blend that the etherification reaction can achieve a 40% olefin conversion and a monoalkyl ether selectivity of greater than 75% while also not being cost prohibitive. CE1 demonstrates that the etherification reaction can be carried out with no solvents, but the olefin conversion does not meet the target 40% or greater. CE2 and CE3 demonstrate that non-oxygenated solvent use alone is unable to concurrently meet the olefin conversion and monoalkyl ether selectivity targets. CE4-CE6 demonstrate that the oxygenated solvents are able to achieve the target olefin conversion and monoalkyl ether selectivity requirements, but as stated before use of only the oxygenated solvents would prove too costly for commercial implementation. IE1-IE8 demonstrates that despite the inclusion of the non-oxygenated solvent at a 50:50 ratio with the oxygenated solvent, the etherification reaction is able to achieve the olefin conversion and monoalkyl ether selectivity targets. IE4, IE9 and CE8 demonstrate that non-oxygenated solvents can be incorporated into the solvent blend at a weight percentage range of 10 wt % to 70 wt % based on a total weight of the solvent blend. IE10 and IE11 demonstrate that the reaction time, ethylene glycol to 1-dodecene ratio and the total solvent concentration can be varied while still achieving the olefin conversion and monoalkyl ether selectivity targets.

What is claimed is:

1. A method of generating an alkylene glycol monoalkyl ether, comprising the steps of:
contacting an olefin, an alcohol, a metallosilicate catalyst, and a solvent blend, wherein the solvent blend comprises from 30 wt % to 90 wt % of an oxygenated solvent based on a total weight of the solvent blend and 10 wt % to 70 wt % of a non-oxygenated solvent based on a total weight of the solvent blend; and
wherein the oxygenated solvent is selected from one or more of cresols, o-cresol, m-cresol, p-cresol, phenol, guaiacol, methyl benzoate, dimethyl phthalate, diglyme, triglyme, diethyl phthalate, dibutyl phthalate, ethyl benzoate, trimethyl 1,2,4-benzenetricarboxylate, glyme, and dimethoxybenzene;
wherein the non-oxygenated solvent is selected from one or more of xylenes, m-xylene, p-xylene, o-xylene, benzene, toluene, hexane, heptane, octane, nonane, undecane, dodecane, tridecane, tetradecane, pentadecane, hexadecane, cyclohexane, cycloheptane, cyclooctane, linear C₆ to C₁₆ alkanes, and cyclic C₆ to C₁₆ alkanes;
generating the alkylene glycol monoalkyl ether.

2. The method of claim 1, wherein the metallosilicate catalyst is an aluminosilicate compound having a crystal lattice that has had one or more metal elements substituted in the crystal lattice for a silicon atom.

3. The method of claim 1, wherein the olefin comprises a C₁₂-C₁₄ alpha-olefin.

4. The method of claim 1, wherein the alcohol is monoethylene glycol, diethylene glycol, glycerol or combinations thereof.

5. The method of claim 1, wherein an alcohol to olefin molar ratio is from 0.5 to 15.

6. The method of claim 1, wherein the solvent blend is from 10 wt % to 80 wt % of the total weight of the combined olefin, alcohol, and solvent blend.

7. The method of claim 1, wherein the solvent blend comprises from 40 wt % to 60 wt % of the non-oxygenated solvent based on a total weight of the solvent blend.

8. (canceled)

9. (canceled)

10. The method of claim 1, wherein the non-oxygenated solvent is xylenes and the oxygenated solvent is cresols.
